(19) United States
(12) Patent Application Publication
(10) Pub. No.: US 2020/0330640 A1
(43) Pub. Date: Oct. 22, 2020

(54) ION GENERATOR, APPARATUS, METHOD FOR PROVIDING CLIMATE-CONTROLLED SPACE, METHOD OF STRESS REDUCTION, METHOD OF IMPROVING DEGREE OF CONCENTRATION, AND METHOD OF IMPROVING DEGREE OF COMFORT

(71) Applicant: SHARP KABUSHIKI KAISHA, Sakai City, Osaka (JP)

(72) Inventors: HIROKAZU FUNAMORI, Sakai City, Osaka (JP); SATOHIKO YAMAMOTO, Sakai City, Osaka (JP); KEITARO YAMADA, Sakai City, Osaka (JP); YUKO MATSUDA, Sakai City, Osaka (JP); HIROKI NANJO, Sakai City, Osaka (JP)

(21) Appl. No.: 16/765,406

(22) PCT Filed: Feb. 15, 2018
(86) PCT No.: PCT/JP2018/005293

§ 371 Date: May 19, 2020

(30) Foreign Application Priority Data
Nov. 28, 2017 (JP) 2017-228285

(51) Int. Cl. A61L 9/22; H01T 23/00

(52) U.S. Cl. CPC A61L 9/22; H01T 23/00

(57) ABSTRACT

Provided is a favorable effect on the mental state of a user who are in a target space that is to undergo climate-control, through ion release. An ion generator (100) causes the target space to have an ion density of 50.000 or more ions/cm³.

### TECHNICAL FIELD

One aspect of the present invention relates to anion generator that releases ions into a target space that is to undergo climate-control, and the aspect relates to other things.

### BACKGROUND ART

Developments and improvements have been made in an apparatus for providing a comfortable space. For instance, Patent Document 1 below discloses a mist generator that releases mists containing chemical substances, such as a fragrance material, a medical chemical, and a deodorizer. Such an apparatus is used in order to give healing to a user.

### CITATION LIST

### Patent Literature

Patent Document 1: Japanese Patent No. 4774040 (registered on Jul. 1, 2011)

### SUMMARY OF INVENTION

### Technical Problem

Like aromatherapy and other treatments, it has been conventionally known that some fragrant ingredients bring a mental effect (e.g., relaxation) to a user, but it has been unknown that an ion generator, one of climate-controlling apparatuses conventionally used, brings this effect. It is an object of one aspect of the present invention to achieve an ion generator and other things capable of having, through ion release, a favorable effect on the mental state of a user who are in a target space that is to undergo climate-control.

### Solution to Problem

To solve the above problem, one aspect of the present invention provides an ion generator that releases ions into a target space that is to undergo climate-control. The ion generator causes the target space to have an ion density of 50,000 or more ions/cm³.

To solve the above problem, another aspect of the present invention provides a method for providing a climate-controlled space by using an ion generator. The method includes a step of releasing ions from the ion generator into the climate-controlled space, thus causing the climate-controlled space to have an ion density of 50,000 or more ions/cm³.

### Advantageous Effect of Invention

Theses aspects of the present invention can have, through ion release, a favorable effect on the mental state of a user who are in a target space that is to undergo climate-control.

### BRIEF DESCRIPTION OF DRAWINGS

FIG. 1 is a schematic cross-sectional view of the configuration of an ion generator according to an embodiment of the present invention.

FIG. 2 is a perspective view of an electric-discharge device that is included in the ion generator.

FIG. 3 is a cross-sectional view of the vicinity of an electric-discharge portion of the electric-discharge device shown in FIG. 2.

FIG. 4 is graphs showing the results of an experiment regarding indexes “concentrated” and “stressed”.

FIG. 5 is graphs showing the results of an experiment regarding the indexes “stressed” and “concentrated” in rooms having ion densities different from each other.

FIG. 6 is a graph showing the value of the index “stressed” before the experiment and one minute after the start of meditation.

FIG. 7 is a graph showing the value of an index “comfortable” before the experiment and one minute after the start of meditation.

FIG. 8 is graphs showing the value of the index “stressed” one minute after the start of meditation and five minutes after the start of the meditation.

FIG. 9 is graphs showing the value of the index “concentrated” one minute after the start of a task and 10 minutes after the start of the task.

### DESCRIPTION OF EMBODIMENTS

### First Embodiment

[Configuration of Ion Generator]

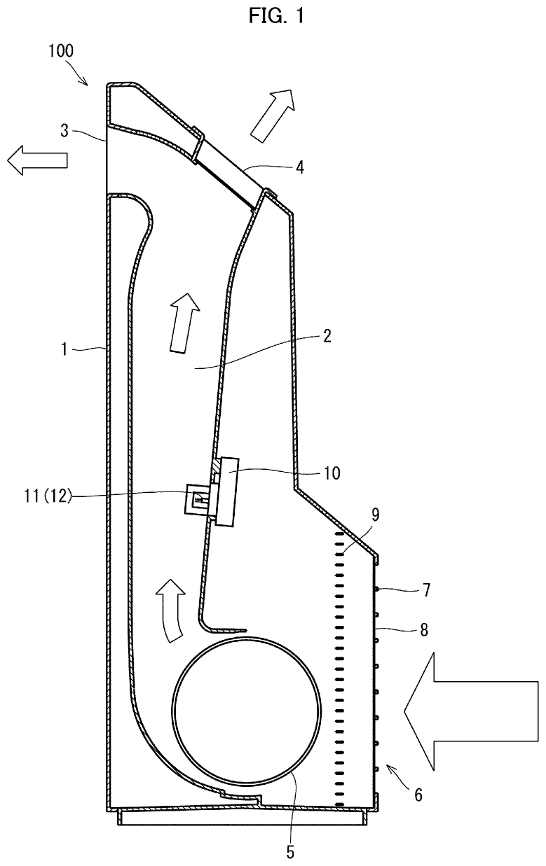

The configuration of an ion generator according to the present embodiment will be described with reference to FIG. 1. FIG. 1 is a schematic cross-sectional view of the configuration of an ion generator 100. As shown in the drawing, the exterior of the ion generator 100 is formed by a casing 1. The front surface of the ion generator 100 is on the left side of the drawing, and the back surface of the ion generator 100 is on the right side of the drawing. The ion generator 100 in the present embodiment is an air purifier capable of ion release.

The casing 1 has a duct 2 inside, which is an air path. The upper end of the duct 2 is branched into two paths. One of the paths is connected to a first outlet 3 opening to the front surface of the ion generator 100, and the other is connected to a second outlet 4 opening to the upper surface of the ion generator 100. It is noted that at least one outlet needs to be provided. The duct 2 is provided with an air blowing device 5 near its lower end. The duct 2 is also provided with an electric-discharge device (i.e., ion generating unit) 10 on its wall.

The electric-discharge device 10 generates active species, including ions (hereinafter merely referred to as ions and other things), through electric discharge. The electric-discharge device 10 includes electric-discharge portions 11 and 12. In the example of the drawing, the electric-discharge device 10 is disposed in the middle in the up-down direction of the duct 2 and on the wall on the back surface of the ion generator 100. The electric-discharge device 10 may be disposed anywhere as long as it is provided in such a manner that the electric-discharge portions 11 and 12 are exposed to the air path. Nevertheless, the electric-discharge portions 11 and 12 are desirably disposed in a location where as much wind as possible is blown, in order to increase the amount of ions and other things discharged by the ion generator 100. Placing these portions in such a location successfully reduces ions that vanish as a result of mutual attraction between positive and negative ions, among positive and negative ions that have been generated. This allows more ions and other things to spread into each and every corner of a target space that is to undergo climate-control.

The air blowing device 5 generates an air current, and is located between the lower end of the duct 2 and an inlet 6. The inlet 6 is disposed below on the back surface of the ion generator 100. Attached to the inlet 6 is a lattice-shaped grille 7 with a filter 8 attached thereto. Disposed between the filter 8 and the air blowing device 5 is a fan guard 9 that protects the air blowing device 5 from, for instance, foreign matters.

Operating the air blowing device 5 takes in air outside the ion generator 100, from the inlet 6 through the filter 8 into the inside of the duct 2, as denoted by the white arrows in the drawing. The air taken in passes through the inside of the duct 2, thus discharging from the first outlet 3 and second outlet 4. Since the electric-discharge device 10 generates ions and other things, the air that goes out from the first outlet 3 and second outlet 4 contains the ions and other things. The ion generator 100 in this way provide a space exterior to the ion generator 100, with ions and other things distributed thereto. The ion generator 100 enables the target space to have a high ion density when compared to a conventional ion generator. The details will be described later on.

[Configuration of Electric-Discharge Device]

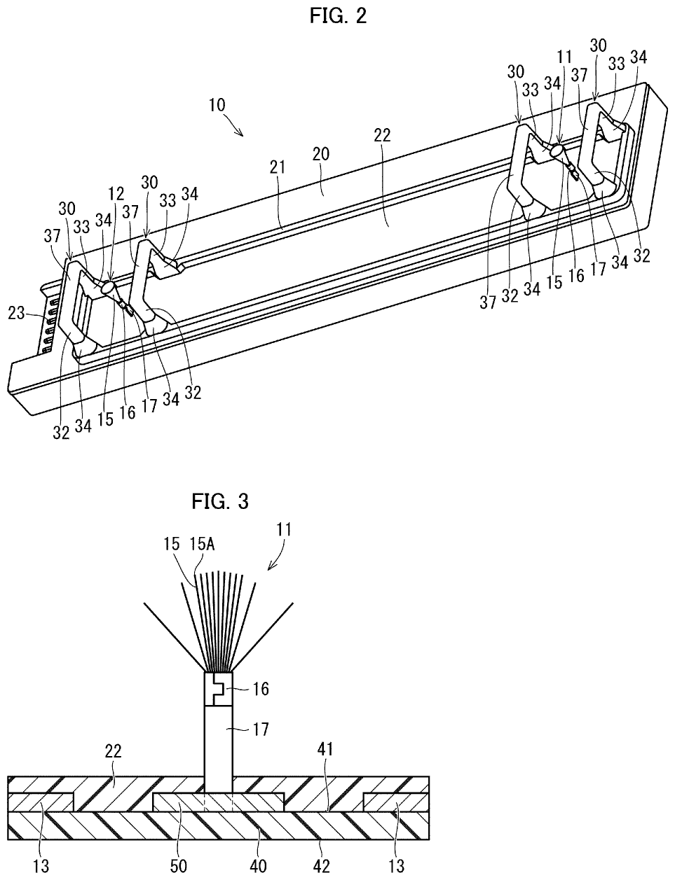

FIG. 2 is a perspective view of the electric-discharge device 10. The electric-discharge device 10 includes the electric-discharge portions 11 and 12 on a casing 20. More specifically, the casing 20 has a wall 21; in addition, the region defined by the wall 21 is filled with insulating resin 22; in addition, the electric-discharge portions 11 and 12 protrude from the resin 22. The electric-discharge device 10 is disposed in such a manner that its longer-side direction (i.e., direction where the electric-discharge portions 11 and 12 are aligned) is perpendicular to the direction of an air current so that the air current within the duct 2 touches the electric-discharge portions 11 and 12 equally.

The electric-discharge portions 11 and 12 generate active species, such as ions. By way of example, the present embodiment describes that positive ions are generated from the electric-discharge portion 11, and that negative ions are generated from the electric-discharge portion 12. Herein, the positive ions are clusters of ions consisting of multiple water molecules in clusters around hydrogen ions (H⁺). These positive ions are expressed as H⁺ (H₂O)ₘ, where m is any integer that equals or exceeds zero. Moreover, the negative ions are clusters of ions consisting of multiple water molecules in clusters around oxygen ions (O₂⁻). These negative ions are expressed as O₂⁻(H₂O)ₙ, where n is any integer that equals or exceeds zero. When the positive and negative ions are released into a room, both ions surround airborne mold germs and viruses, thus causing chemical reactions mutually on their surfaces. The airborne mold germs and other things are removed by the action of the hydroxyl radicals (.OH) of active species generated in these reactions.

If the electric-discharge portions 11 and 12 are too close to each other, many of the positive and negative ions generated are neutralized to vanish. Let the distance between the electric-discharge portions 11 and 12 of the electric-discharge device 10 in the present embodiment be 100%. Accordingly, for 80% of the distance, the amount of ions decreases by about 10%. Moreover, if the electric-discharge portions 11 and 12 are too distant from each other, the amount of ions that goes out reduces. Let the distance between the electric-discharge portions 11 and 12 be 200%. Accordingly, the amount of ions decreases by about 20%. For the reasons set forth above, the distance between the electric-discharge portions 11 and 12 is desirably regulated so that a desired amount of ions goes out.

The electric-discharge portion 11 is a brush-shaped electrode that includes multiple conductors 15, a base 16 bundling the conductors 15, and a support 17 supporting the conductors 15 on the casing 20. The electric-discharge portion 12 is configured similarly. Upon voltage application to the electric-discharge portion 11, the conductors 15 have the same polarity, thus electrically repelling one another to form into a brush with its bristles splayed. Consequently, the use of the conductors 15 enables ions and other things to spread into a wide range, thus achieving a higher density of ions than the use of an electrode having a needle-shaped distal end. It is noted that the electric-discharge portions 11 and 12 (i.e., electrodes) may have any shape that achieves a desired ion density; examples of such a shape include a needle, bar, line, fiber, and plane.

The electric-discharge device 10 includes electrode protectors 30, and includes a connector 23 connecting a wire for supplying electricity to the electric-discharge device 10 and for controlling the electric-discharge device 10. The electrode protectors 30 protect the electric-discharge portions 11 and 12 from contact with foreign matters and are used in pairs. To be specific, a pair of electrode protectors 30 sandwiching the electric-discharge portion 11 from both sides in the side-to-side direction protects the electric-discharge portion 11. In addition, another pair of electrode protectors 30 sandwiching the electric-discharge portion 12 from both sides in the side-to-side direction protects the electric-discharge portion 12.

Each electrode protector 30 includes an upstream column 32, a downstream column 33, and a beam 37. The upstream column 32 is upstream of the electric-discharge portion 11 or 12. The downstream column 33 is downstream of the electric-discharge portion 11 or 12. The electrode protector 30 is in the form of an arch with the beam 37 supported by the upstream column 32 and downstream column 33. The upstream column 32 and the downstream column 33 have their proximal ends in the form of trapezoids, which are roots 34.

The upstream column 32 and downstream column 33 are longer than the electric-discharge portion 11 or 12. Accordingly, a foreign matter, if any, that approaches the electric-discharge portion 11 or 12 is blocked by the beam 37, thus failing to come into contact with the electric-discharge portion 11 or 12. Moreover, the region defined by the upstream column 32, downstream column 33, and beam 37 is an opening; accordingly, the electric-discharge portion 11 or 12 is visible through the opening when the electric-discharge device 10 is viewed from its side, as shown in FIG. 1.

Providing the trapezoidal roots 34 turns their surfaces into air-current guiding surfaces that guide an air current toward the electric-discharge portion 11 or 12. These surfaces guide the air current passing through the pair of electrode protectors 30, to the electric-discharge portion 11 or 12. This allows the ions and other things generated at the electric-discharge portion 11 or 12 to go out immediately, thereby preventing reduction in the ion density resulting from contact with the wall of the duct 2 and other factors. In addition, the generated ions and other things diffuse immediately, thereby preventing reduction in the ion density resulting from neutralization of the positive and negative ions. Providing the electrode protectors 30 with the roots 34 in this way improves the efficiency of generating ions and other things, thereby successfully creating a space having ions and other things distributed densely.

The air-current guiding surfaces may be formed in any manner, and can be achieved by a configuration other than the roots 34. For instance, the upper end of each upstream column 32 (i.e., an end connected to the beam 37) may have a shape that widens along with approach to the beam 37. In this case, the widened portion serves as an air-current guiding surface and guides the air current to the electric-discharge portion 11 or 12. Alternatively, the pair of electrode protectors 30 on the casing 20 may be placed in a different manner, for instance, the pair of electrode protectors 30 may be disposed in such a manner that the distance between the upstream columns 32 is smaller than the distance between the downstream columns 33. Consequently, the side surfaces of the upstream columns 32 and the side surfaces of the beams 37 can serve as air-current guiding surfaces.

[Configuration of Electric-Discharge Portion]

FIG. 3 is a cross-sectional view of the vicinity of the electric-discharge portion 11 of the electric-discharge device 10 shown in FIG. 2. The electric-discharge portion 11 is secured to a plate board 40. The board 40 has a surface 41 provided with an induction electrode 13 and with a repulsive electrode 50. Although not shown, the board 40 has a back surface 42 secured to the casing 20. The induction electrode 13 and repulsive electrode 50 are sealed by the resin 22 and are thus insulated.

The induction electrode 13 is an annular electrode that surrounds the electric-discharge portion 11 in top view. The electric-discharge portion 11 is located at the center of the annular ring. Applying a high positive pulse to the induction electrode 13 discharges electricity from the electric-discharge portion 11 (to be more specific, from distal ends 15A of the conductors 15), thus generating positive ions. It is noted that the induction electrode 13 may have any shape other than the annular shape.

The repulsive electrode 50 is an annular electrode that surrounds the support 17 in top view. The support 17 (as well as the electric-discharge portion 11) is located at the center of the annular ring. The repulsive electrode 50 is provided for achieving a higher ion density, and receives a voltage having the same polarity as the induction electrode 13 (i.e., a positive voltage in this example).

As described above, voltage application to the induction electrode 13 generates positive ions from the distal ends 15A of the conductors 15. The positive ions move along lines of electric force that extend from the distal ends 15A of the conductors 15 toward the induction electrode 13. When a voltage having the same polarity as that of the induction electrode 13 is applied to the repulsive electrode 50, these lines of electric force form a shape that avoids the repulsive electrode 50 and thus makes a large turn around the repulsive electrode 50. The positive ions thus tend to diffuse toward the upper part of the repulsive electrode 50 so as to move away from the repulsive electrode 50. The positive ions hence reduce positive ions that are collected by the induction electrode 13 and its surroundings (i.e., the board 40 and resin 22), thereby allowing more positive ions to spread into the air current than ever before.

It is noted that the repulsive electrode 50 may have any shape other than the annular shape. The foregoing has described the electric-discharge portion 11. The electric-discharge portion 12 is configured similarly. That is, like the electric-discharge portion 11, an induction electrode and a repulsive electrode are provided for the electric-discharge portion 12. In addition, applying a negative voltage to the induction electrode and repulsive electrode of the electric-discharge portion 12 allows more negative ions to spread into the air current.

[Regulation of Electric Discharge]

As described above, voltage application to the induction electrode 13 discharges electricity from the electric-discharge portion 11, thus releasing positive ions. Likewise, negative ions is released from the electric-discharge portion 12. In order to be higher than ever before, the ion density is desirably regulated in such a manner that electric discharge per unit time outnumbers conventional, electric discharge per unit time.

[Output Switching]

The ion generator 100 may be capable of output switching. For instance, the ion generator 100 may include an input unit (e.g., a button and a touch panel) on the surface of the casing 1 for instance. This input unit receives a user's input operation. It is noted that the input unit may receive an operation that is input remotely via a remote control or other means. It is also noted that in accordance with the input operation received by the input unit, the ion generator 100 may gradually or continuously increase or decrease the amount of ions to be released. For instance, the ion generator 100 may regulate the amount of ions to be released, at three levels: low, middle, and high. In this case, it is desirable to provide an output level that enables the target space to have an ion density of 50,000 or more ions/cm³, and to provide an output level that enables the target space to have an ion density of less than 50,000 ions/cm³. For instance, at the low level, the target space may have an ion density of about 12,500 ions/cm³. Further, at the middle level, the target space may have an ion density of about 25,000 ions/cm³. Still further, at the high level, the target space may have an ion density of about 50,000 ions/cm³. It is noted that the ion generator 100 enables the target space to have a positive-ion density of 50,000 or more positive ions/cm³ and a negative-ion density of 50,000 or more negative ions.

The target space is caused to have an ion density of 50,000 or more ions/cm³. This ion density has been proven to achieve an effect that can never achieved by a conventional ion generator. The details will be described in a second embodiment and the subsequent embodiments. Providing the aforementioned output levels enables the user to achieve this effect, as necessary, by switching the output levels. It is noted that operation modes may be specified that achieve such an effect. In this case, the user switches between the operation modes, thereby achieving the aforementioned effect as necessary.

Any configuration may be provided to achieve an ion density of 50,000 or more ions/cm³. For instance, the ion density of 50,000 or more ions/cm³ can be achieved by omitting one or more of the various configurations (i.e., the control of electric discharge, the repulsive electrodes, the air-current guiding surfaces, the regulation of the distance between the electric-discharge portions 11 and 12, and the brush-shaped electrodes) for improvement in the ion density. Among these configurations, the regulation of the distance between the electric-discharge portions 11 and 12 contributes to improvement in the ion density very much; hence it is desirable to use the electric-discharge device 10 with at least the distance between the electric-discharge portions 11 and 12 regulated suitably.

### Second Embodiment

Another embodiment of the present invention will be described. For the sake of convenience in description, components whose functions are the same as those described in the foregoing embodiment are denoted by the same signs and will not be elaborated upon. This holds true for a third embodiment.

The ion generator 100 according to the present embodiment, which has various configurations for increasing ion density, can provide a space having a very high ion density of 50,000 or more ions/cm³. Such a high density can be never achieved by a conventional ion generator. The inventors of the present invention conducted an experiment to find that such a space having a high ion density had a favorable effect on the mental state of a person who were in the space. The present embodiment describes the details of the experiment and its results. It is noted that the aforementioned space having a high ion density cannot be provided by merely combining multiple conventional electric-discharge devices, because positive and negative ions neutralize to vanish upon contact, because these ions vanish upon contact with an obstacle, and for other reasons.

[Details of Experiment]

The inventors prepared a room having an ion density of 50,000 or more ions/cm³ generated by the ion generator 100 (hereinafter, the room is referred to as a room with ions). The inventors also prepared a room having no ions generated by the ion generator 100 (hereinafter, the room is referred to as a room without ions). The actual measurement of positive-ion density was 62,000 ions/cm³ at 120 cm above the floor in the middle of the room. In addition, the actual measurement of negative-ion density was 53,000 ions/cm³ at 120 cm above the floor in the middle of the room. In this way, the room in this experiment example has a positive-ion density of 50,000 or more positive ions/cm³ and a negative-ion density of 50,000 or more negative ions cm³.

The inventors then asked male and female subjects in their twenties to do a predetermined process in each room to obtain their brain-wave data at that time. Specifically, the inventors measured the brain wave of each subject before the experiment. The inventors then asked the subjects to enter the room with ions or the room without ions, be seated, and take a deep breath. The inventors then measured their brain waves. The brain waves at this time were referred to as brain waves at the time of room entrance. Thereafter, the inventors asked the subjects to do a predetermined task (i.e., herein, the Kraepelin test), and measured their brain waves during the task. Then, the inventors measured the brain waves of the subjects again while the subjects were resting after the task. The brain waves at this time were referred to as brain waves after the task.

The inventors prepared three rooms with ions and three rooms without ions. The inventors placed the ion generator 100 in the first room with ions, and then had this room used by 11 subjects. In addition, the inventors placed a dummy of the ion generator 100 (i.e., apparatus that generates no ions) in the first room without ions, and then had this room used by 11 subjects.

In the second room with ions, the inventors placed an air conditioning apparatus that includes the electric-discharge device 10 and can generate as many ions as the ion generator 100, and the inventors had this room used by 15 subjects. In addition, the inventors placed an air conditioning apparatus (i.e., apparatus that generates no ions) in the second room without ions, and had this room used by 15 subjects.

The inventors placed the ion generator 100 in the third room with ions, and had this room used by 23 subjects. The third room with ions was wider than the first room with ions. Moreover, the inventors placed a dummy of the ion generator 100 in the third room without ions, and had this room used by 23 subjects. The third room without ions was wider than the first room without ions. The inventors obtained brain-wave data of 49 subjects in total who were in the rooms with ions, and obtained brain-wave data of 49 subjects in total who were in the rooms without ions.

Using the measured brain waves, the inventors calculated indexes each indicating the mental state of the subjects. In this experiment, the inventors used a headgear-like simplified electroencephalograph. These indexes are each established, by extracting characteristic points for each frequency of the brain wave, followed by combining the extracted characteristic points. Specifically, using the brain-wave data obtained through the measurement, the inventors calculated an index indicating a numeric degree that falls under a mental state “concentrated” (here, a larger numeric degree equals higher concentration). Likewise, the inventors calculated indexes indicating numeric degrees that fall under respective mental states: “stressed”, “comfortable”, “favorite” (i.e., the degree of favorableness to a target space that is to undergo climate-control) and “interested”. How to calculate these indexes and the principle of the calculation, which are detailed in Japanese Patent Application Laid-Open No. 2015-109964 and other published documents, will not be elaborated upon.

[Experiment Results]

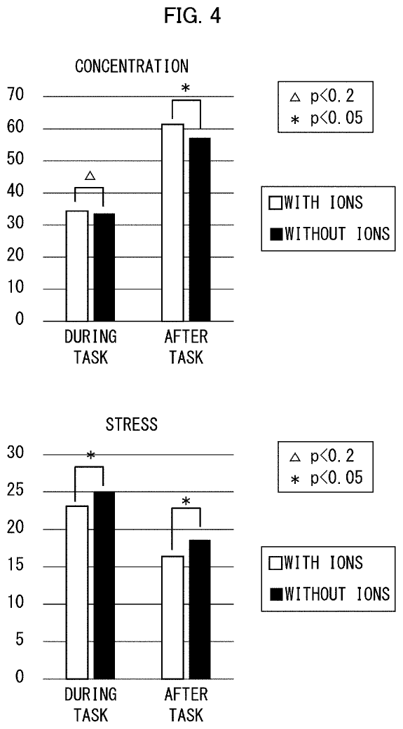

FIG. 4 is graphs showing the results of the experiment regarding the indexes “concentrated” and “stressed”. As shown in the drawing, the experiment brought a result that the index “concentrated” after the task was significantly higher in the rooms with ions than that in the rooms without ions (herein, a p-value of less than 0.05). In other words, the subjects in the rooms with ions became more concentrated after the task than those in the rooms without ions. The experiment also brought a result that even during the task, the index “concentrated” was possibly, significantly higher in the rooms with ions than that in the rooms without ions (herein, a p-value of less than 0.2).

The experiment also brought a result that the index “stressed” during and after the task was significantly lower in the rooms with ions than that in the rooms without ions (herein, a p-value of less than 0.05). In other words, the subjects in the rooms with ions were less under stress during and after the task than those in the rooms without ions.

As described above, the subjects in the rooms with ions were more concentrated and less under stress during the task. The inventors have accordingly concluded that a high concentration of ions less put a mental load on the subjects, thereby allowing the subjects to do the task while being relaxed and concentrated.

Those in the rooms with ions became concentrated greatly and were less under stress after the task. The inventors have accordingly concluded that a high concentration of ions can accelerate the reset speed of the subjects (i.e., the speed at which the subjects recover from stress caused by the task). The acceleration in the reset speed is promising to enhance a desire (i.e., motivation) to attain the task.

For the sake of comparison, the inventors conducted a similar experiment in a room where negative ions were generated by a conventional ion generator (i.e., ion generator that generates no positive ions). The comparative experiment brought a result that no significant change in the index “favorite” was found between before and after room entrance. The comparative experiment also brought a result that the index “stressed” during the task reduced greatly when compared to that after room entrance. The comparative experiment also brought a result that no significant change in the index “stressed” was found between the post-room-entrance and during the task.

[Summary of Experiment Results]

The experiment brought a result that during the meditation and during and after the task, the index “stressed” was lower and the index “comfortable” were higher in the rooms with ions than those in the rooms without ions. The experiment also brought a result that during the meditation and during and after the task, the index “favorite” was higher in the rooms with ions than that in the rooms without ions. Reference is made to the difference in the value of the index “favorite” between the rooms with ions and the rooms without ions. The p-value was less than 0.05 during the meditation (i.e., there was a significant difference) and was less than 0.1 during and after the task (i.e., there was a significant tendency).

The rate of change in the index “concentrated” between before and after the task was large in the rooms with ions. Reference is made to the difference in the value of the index “concentrated” after the task between the rooms with ions and the rooms without ions. As shown in FIG. 4, the p-value was less than 0.05 (i.e., there was a significant difference).

[Relationship Between Ion Density and Stress]

The inventors also conducted an experiment regarding the relationship between ion density and stress, and regarding the relationship between ion density and the degree of concentration. Specifically, the inventors prepared rooms having an ion density half the ion density in the aforementioned experiment (i.e., about 25,000 ions/cm³), followed by measuring the brain waves of the individual subjects at the time of room entrance as well as during and after the task through procedures similar to those in the aforementioned experiment, followed by calculating the indexes “stressed” and “concentrated” from the measured results.

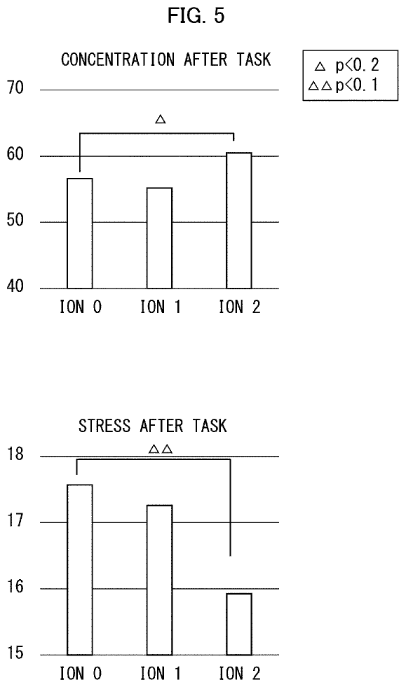

FIG. 5 is graphs showing the results of the experiment regarding the indexes “stressed” and “concentrated” in the rooms having ion densities different from one another. Specifically, the upper part of FIG. 5 is a bar chart showing a change in the index “concentrated” before and after the task for each of Ions 0 to 2. As shown in the drawing, a room having an ion density of about 50,000 ions/cm³ can significantly increase the index “concentrated” after the task, when compared to that in a room without ions (herein, a p-value of less than 0.2). The experiment brought a result that no significant difference was found between the room without ions and a room with ions having an ion density of about 25,000 ions/cm³. In this way, the experiment has revealed that an ion density of about 50,000 ions/cm³ increases the index “concentrated” after the task. This increase can be never achieved by an ion density of about 25,000 or less ions/cm³.

The lower part of FIG. 5 is a bar chart showing a change in the index “stressed” before and after the task for each of Ions 0 to 2. Here, Ion 0 indicates the experiment result in the room without ions. Further, Ion 1 indicates the experiment result in the room with ions having an ion density of about 25,000 ions/cm³. Still further, Ion 2 indicates the experiment result in the room with ions having an ion density of about 50,000 ions/cm³.

As shown in the graph, an ion density of about 50,000 ions/cm³ decreased the index “stressed” after the task when compared to that in the room without ions, and had a significant tendency (herein, a p-value of less than 0.1). Meanwhile, no significant difference was found between the room without ions and the room with ions having an ion density of about 25,000 ions/cm³. In this way, the experiment has revealed that an ion density of about 50,000 ions/cm³ promotes reduction in the index “stressed” after the task. This promotion can be never achieved by an ion density of about 25,000 or less ions/cm³.

Referring to ion density in nature, there are at most 1,000 ions/cm³ even in a forest, which will be described later on. Even an ion density of 25,000 ions/cm³ is considerably higher than the ion density in nature. Accordingly, even those skilled in the art are seemed to have difficulty in predicting that an ion density of about 50,000 ions/cm³, further higher than an ion density of 25,000 ions/cm³, can achieve an effect that can be never achieved by the ion density of 25,000 ions/cm³. In particular, those skilled in the art are further seemed to have difficulty in predicting that the promotion of reduction in the index “stressed” and the promotion of increase in the index “concentrated” can be achieved by an ion density of about 50,000 ions/cm³.

In order to compare the effect with that of the ion generator 100, the inventors measured brain waves in a forest, which is commonly said to make a person feel relaxed. Specifically, the inventors asked a total of 12 male and female subjects in their twenties to sixties, to gather in Tokyo. At this point, the inventors measured the brain waves of the individual subjects before conducting the experiment. The inventors then asked the subjects to move to the back hill of No-nin-ji Temple in Hanno City, Saitama, and to do 10-minute meditation. The inventors then measured the brain waves of the individual subjects during the meditation. At the back hill of No-nin-ji Temple at the time of the experiment, the temperature was 25° C., the humidity was 67% RH, the weather was cloudy, occasionally sunny, the wind velocity was 0.2 m/s, the density of positive ions was 410 ions/cm³, and the density of negative ions was 780 ions/cm³.

Furthermore, on the next day of the experiment at the back hill of No-nin-ji Temple, the inventors asked the subjects to move to Agatsumakyou Valley in Saitama, and conducted a similar experiment there. At Agatsumakyou Valley at the time of the experiment, the weather was 23° C., the humidity was 56% RH, the weather was sunny, occasionally cloudy, the wind velocity was 0.4 m/s, the density of positive ions was 420 ions/cm³, and the density of negative ions was 690 ions/cm³.

Based on the brain waves measured in the aforementioned manner, the inventors then calculated indexes in numeral indicating degrees that fall under respective mental states “stressed”, “concentrated”, “comfortable”, “favorite”, and “interested”, as is the case with the individual examples described above.

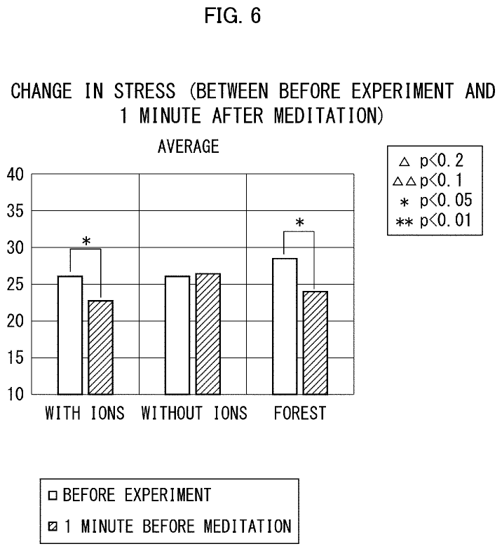

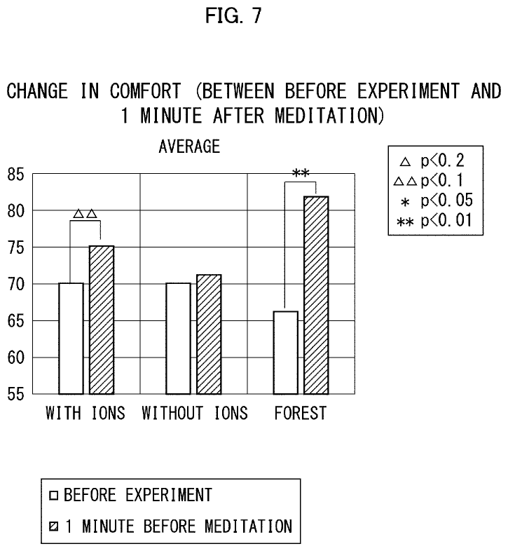

The back hill of No-nin-ji Temple and Agatsumakyou Valley are in common in the following points: (1) Although both are surrounded by trees, visibility is good to a certain extent, and (2) There are waterfronts nearby, such as a river and a pond. In the Description, an outdoor region satisfying these conditions is referred to as a forest. The experiments brought a result that in these forests, the index “stressed” decreased and the index “comfortable” increased. The detailed result will be described later on with reference to FIGS. 6 and 7. This result are similar to that in the rooms with ions.

[Change in Index “Stressed” (i.e., Comparison Between Before Experiment and One Minute after Meditation Start)]

FIG. 6 is a graph showing the value of the index “stressed” before the experiments and showing the value of the index “stressed” one minute after the start of meditation. The value of the index “stressed” is an average of multiple subjects. As shown in the graph, in the room without ions (i.e., nothing), no significant change in the index “stressed” was found between before the experiment and one minute after the meditation start. In contrast, in the rooms with ions and the forests, the index “stressed” decreased. To be more specific, in the rooms with ions, the index “stressed” decreased by 12.7%, and the p-value was less than 0.05 (i.e., there was a significant difference). In the forests, the index “stressed” decreased by 15.8%, and the p-value was less than 0.05 (i.e., there was a significant difference). In this way, the change in the index “stressed” between before the experiment and one minute after the meditation start is similar between the rooms with ions and the forests. In addition, the rate of change in the room with ions is as great as that in the forests.

In this way, the rooms with ions reduce the index “stressed” of the subjects, as is the case with the forests. In other words, the aforementioned experiments based on the measured brain waves have demonstrated that the ion generator 100 allows a user to enjoy, while being in a target space that is to undergo climate-control, a mental effect, i.e., stress reduction that is similarly enjoyed when the user has moved from a city to a forest. Thus, the ion generator 100 can provide an environment, like a forest, where the user feels less stressed and thus feels relaxed.

[Change in Index “Comfortable” (i.e., Comparison Between Before Experiment and One Minute after Meditation Start)]

FIG. 7 is a graph showing the value of the index “comfortable” before the experiments and showing the value of the index “comfortable” one minute after the start of meditation. As is the case with FIG. 6, the value of the index “comfortable” is an average of multiple subjects. As shown in the graph, in the room without ions (i.e., nothing), no significant change in the index “comfortable” was found between before the experiment and one minute after the meditation start. In contrast, in the rooms with ions and the forests, the index “comfortable” increased. To be more specific, in the rooms with ions, the index “comfortable” increased by 7%, and the p-value was less than 0.1 (i.e., there was a significant tendency). Further, in the forests, the index “comfortable” increased by 24%, and the p-value was less than 0.01 (i.e., there was a significant difference). In this way, the change in the index “comfortable” between before the experiment and one minute after the meditation start is similar between the rooms with ions and the forests.

In this way, in the rooms with ions, the index “comfortable” of the subjects increased, as is the case with the forests. In other words, the aforementioned experiments based on the measured brain waves have demonstrated that the ion generator 100 allows a user to enjoy, while being in a target space that is to undergo climate-control, a mental effect, i.e., increase in the degree of comfort that is enjoyed when the user has moved from a city to a forest.

### Third Embodiment

The present embodiment describes a further another experiment for verifying how the ion generator 100 affects the mental state of a person.

[Details of Experiment]

In this experiment, the inventors asked individual subjects to do a predetermined task in each of a room with ions and a room without ions, and then acquired their brain-wave data at that time. In this experiment as well, the inventors measured the brain waves in the room with ions. The inventors controlled the air in the room with ions, by using an air conditioning apparatus that includes the electric-discharge device 10.

There was a total of 39 subjects in this experiment, among which 19 were elementary-school males and females in the fifth and sixth grades (i.e., children), and 20 were males and females in their thirties and forties (i.e., adults). The subjects were separated into groups of four to six people. The individual subjects had their brain waves measured for one minute (here, the brain waves were measured before the experiment). Then, the subjects were asked to enter the room with ions and the room without ions on a group basis.

After room entrance, the subjects were asked to sequentially perform 10-minute meditation, take a one-minute rest, and do a 10-minute task (herein, the Kraepelin test). The subjects then had their brain waves measured individually during the meditation and the task. The subjects then had their brain waves measured for one minute while remained at rest after the task. The brain waves at this time were referred to as brain waves after the task.

[Change in Index “Stressed” (i.e., Comparison Between One Minute after Meditation Start and Five Minutes after Meditation Start)]

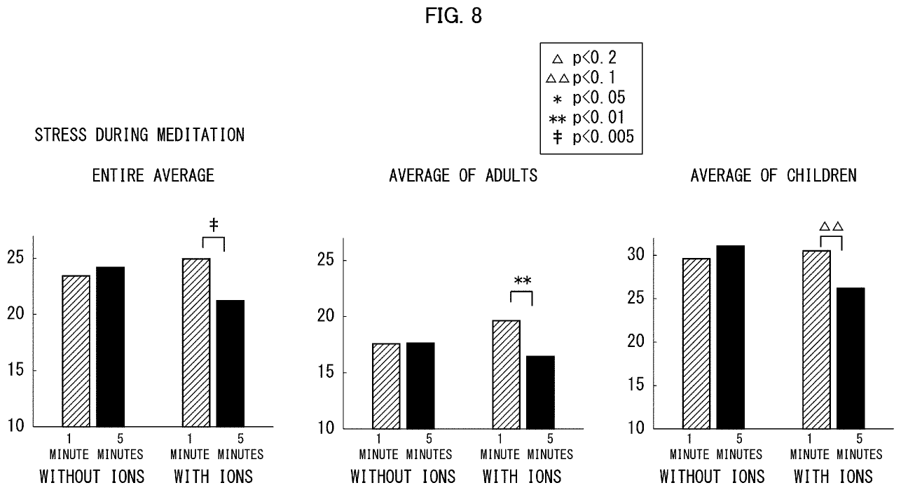

FIG. 8 is graphs showing the value of the index “stressed” one minute after meditation start and five minutes after meditation start. To be more specific, the left part of the drawing is a bar chart showing averages of all the 39 subjects, the middle part of the same is a bar chart showing averages of the adults (in their thirties and forties), and the right part of the same is a bar chart showing averages of the children (in the fifth and sixth grade in elementary school).

Reference is made to the entire averages. The index “stressed” slightly increased between one minute and five minutes after the meditation start in the room without ions. In contrast, the index “stressed” decreased in the room with ions. This decrease was accompanied by a p-value of less than 0.005, and there was a significant difference.

Reference is made to the averages of the adults. No significant difference in the index “stressed” was found between one minute and five minutes after the meditation start in the room without ions. In contrast, the index “stressed” decreased in the room with ions. This decrease was accompanied by a p-value of less than 0.01, and there was a significant difference.

Reference is made to the averages of the children. The index “stressed” slightly increased between one minute and five minutes after the meditation start in the room without ions. In contrast, the index “stressed” decreased in the room with ions. This decrease was accompanied by a p-value of less than 0.1, and there was a significant tendency.

In this way, the index “stressed” of both the adults and children decreased between one minute and five minutes after the meditation start in the room with ions. These experiment results support an effect of reducing stress in the room with ions.

[Change in Index “Concentrated” (i.e., Comparison Between One Minute after Task Start and 10 Minutes after Task Start)]

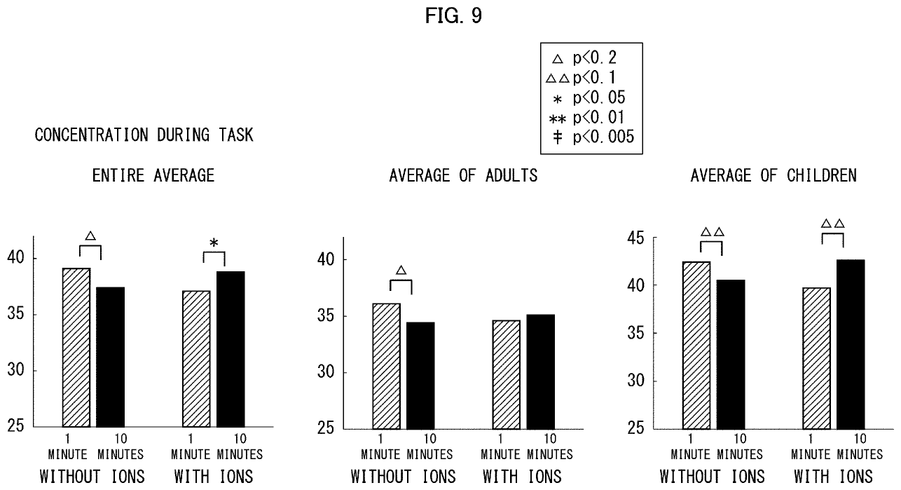

FIG. 9 is graphs showing the value of the index “concentrated” one minute after task start and 10 minutes after the task start. To be more specific, the left part of the drawing is a bar chart showing averages of all the 39 subjects, the middle part the same is a bar chart showing averages of the adults (in their thirties and forties), and the right part of the same is a bar chart showing averages of the children (in the fifth and sixth grade in elementary school).

Reference is made to the entire averages. The index “concentrated” decreased between one minute and 10 minutes after the task start in the room without ions. This decrease was accompanied by a p-value of less than 0.2, and there was possibly a significant difference. In contrast, the index “concentrated” increased in the room with ions. This increase was accompanied by a p-value of less than 0.05, and there was a significant difference.

Reference is made to the averages of the adults. The index “concentrated” decreased between one minute and 10 minutes after the meditation start in the room without ions. This decrease was accompanied by a p-value of less than 0.2, and there was possibly a significant difference. In contrast, the index “concentrated” slightly increased in the room with ions.

Reference is made to the averages of the children. The index “concentrated” decreased between one minute and 10 minutes after the meditation start in the room without ions. This decrease was accompanied by a p-value of less than 0.1, and there was a tendency toward a significant difference. In contrast, the index “concentrated” increased in the room with ions. This increase was accompanied by a p-value of less than 0.1, and there was a significant tendency.

In this way, the index “concentrated” of both the adults and children increased between one minute and 10 minutes after the task start in the room with ions. These experiment results support an effect of improving concentration in the room with ions.

[Ion Generator 100]

Based on the foregoing experiment results, the inventors conclude that the ion generator 100 releases ions into a target space that is to undergo climate-control, thus reducing the stress of a person who are in the target space. Further, the inventors conclude that the ion generator 100 releases ions into a target space that is to undergo climate-control, thus improving the degree of concentration of a person who are in the target space. Still further, the inventors conclude that the ion generator 100 releases ions into a target space that is to undergo climate-control, thus improving the degree of comfort of a person who are in the target space.

[Modifications]

The forgoing embodiments have described that the ion generator 100 (i.e., air purifier) that includes the electric-discharge device 10, or the air conditioning apparatus that includes the electric-discharge device 10 provides a target space that is to undergo climate-control having a predetermined ion density (i.e., 50,000 or more ions/cm³) that have favorable effects on the mental state of a user, such as a reduction in stress, an improvement in the degree of concentration, and an improvement in the degree of comfort. These apparatuses are illustrative, and the present invention encompasses any ion generator that enables a target space that is to undergo climate-control, to have this predetermined ion density. For instance, the present invention encompasses an air-conditioning apparatus that includes the electric-discharge device 10 disposed in a vehicle-installed device, and that causes the vehicle interior to have the predetermined ion density. The present invention also encompasses various apparatuses each including the electric-discharge device 10 (e.g., a chair, a desk, bedding, including a bed, and furniture). Moreover, an ion generator and ion generating unit according to one aspect of the present invention needs to be an ion generator that can provide a climate-controlled space having the aforementioned predetermined ion density, and the ion generator and ion generating unit does not have to include the electric-discharge device 10.

A method for providing a climate-controlled space by using the ion generator according to the present invention includes a step of releasing ions from the ion generator into the climate-controlled space, thus causing the climate-controlled space to have the aforementioned predetermined ion density. The climate-controlled space may be not only a conference room and other rooms, but also a living room, bedroom, bathroom, or other rooms that is intended for home use. Alternatively, the climate-controlled space may be a waiting room in a station, hospital, or other facilities, or may be an examination room in a hospital, the interior of a vehicle, or other facilities. Alternatively, the climate-controlled space may be a classroom or self-study room in, for instance, a school or after-hours cram school, or may be a library or other facilities.

As described above, releasing ions from the ion generator 100 into a target space that is to undergo climate-control can reduce the stress of a person who are in the target space. Accordingly, one aspect of the present invention can be also expressed as a method of stress reduction using the ion generator 100. Likewise, releasing ions from the ion generator 100 into a target space that is to undergo climate-control can also improve the degree of concentration and comfort of a person who are in the target space. Accordingly, one aspect of the present invention can be also expressed as a method of improving the degree of concentration by using the ion generator 100, or as a method of improving the degree of comfort by using the ion generator 100.

### Summary

An ion generator (100) according to a first aspect of the present invention releases ions into a target space that is to undergo climate-control. The ion generator causes the target space to have an ion density of 50,000 or more ions/cm³.

This configuration not only achieves an effect (e.g., germ reduction) obtained by a conventional ion generator, but also, for instance, reduces the stress of a user and increases the degree of the user's concentration. These effects have never been proven. This configuration is also promising to increase the degree of comfort and the degree of favorableness (i.e., degree indicating how much a user likes a target space that is to undergo climate-control). This configuration is also promising for a user to enjoy, while being in the target space, a reduction in stress and an increase in the degree of comfort as much as he/she does when having moved from a city to a forest.

An ion generator according to a second aspect of the present invention may, in the first aspect, cause the target space to have a positive-ion density of 50,000 or more positive ions/cm³ and a negative-ion density of 50,000 or more negative ions/cm³.

The experiments conducted by the inventors of the present invention have demonstrated that a configuration where a target space that is to undergo climate-control has a positive-ion density of 50,000 or more positive ions and a negative-ion density of 50,000 or more negative ions achieves the aforementioned effects. Consequently, these effects can be achieved highly probably.

An apparatus according to a third aspect of the present invention includes an ion generating unit (i.e., electric-discharge device 10) that releases ions into a target space that is to undergo climate-control. The ion generating unit causes the target space to have an ion density of 50,000 or more ions/cm³. This configuration achieves effects similar to those in the first aspect.

A fourth aspect of the present invention provides a method for providing a climate-controlled space by using an ion generator. The method includes a step of releasing ions from the ion generator into the climate-controlled space, thus causing the climate-controlled space to have an ion density of 50,000 or more ions/cm³.

This configuration successfully provides a climate-controlled space that, for instance, reduces the stress of a user and increases the degree of the user's concentration. It is noted that such a climate-controlled space that affects mentality can be never provided by a conventional ion generator, and can be provided for the first time by the ion generator according to the present invention.

The present invention encompasses a method of stress reduction. The method includes releasing ions from the ion generator into a target space that is to undergo climate-control, thus reducing the stress of a person who are in the target space. The present invention also encompasses a method of improving the degree of concentration. The method includes releasing ions from the ion generator into a target space that is to undergo climate-control, thus improving the degree of concentration of a person who are in the target space. The present invention also encompasses a method of improving the degree of comfort. The method includes releasing ions from the ion generator into a target space that is to undergo climate-control, thus improving the degree of comfort of a person who are in the target space.

The present invention also encompasses the ion generator (100) that releases the ions into the target space, thus reducing the stress of a person who are in the target space. The present invention also encompasses the ion generator (100) that releases the ions into the target space, thus improving the degree of concentration of a person who are in the target space. The present invention also encompasses the ion generator (100) that releases the ions into the target space, thus improving the degree of comfort of a person who are in the target space.

The present invention is not limited to the aforementioned embodiments. Numerous modifications can be made within the scope of the claims. The present invention also encompasses an embodiment that can be obtained in combination, as necessary, with the technical means disclosed in the respective embodiments. Furthermore, combining technical means disclosed in the respective embodiments can form a new technical feature.

### REFERENCE SIGNS LIST

10 electric-discharge device (ion generating unit)
100 ion generator

1. An ion generator configured to release ions into a target space that is to undergo climate-control,
wherein the ion generator causes the target space to have an ion density of 50,000 or more ions/cm³.

2. The ion generator according to claim 1, wherein the ion generator causes the target space to have a positive-ion density of 50,000 or more positive ions/cm³ and a negative-ion density of 50,000 or more negative ions/cm³.

3. An apparatus comprising
an ion generating unit configured to release ions into a target space that is to undergo climate-control,
wherein the ion generating unit causes the target space to have an ion density of 50,000 or more ions/cm³.

4. A method for providing a climate-controlled space by using an ion generator, the method comprising
a step of releasing ions from the ion generator into the climate-controlled space, thus causing the climate-controlled space to have an ion density of 50,000 or more ions/cm³.

5. A method of stress reduction, comprising
releasing ions from the ion generator according to claim 1 into a target space that is to undergo climate-control, thus reducing a stress of a person who are in the target space.

6. A method of improving a degree of concentration, comprising
releasing ions from the ion generator according to claim 1 into a target space that is to undergo climate-control, thus improving a degree of concentration of a person who are in the target space.

7. A method of improving a degree of comfort, comprising
releasing ions from the ion generator according to claim 1 into a target space that is to undergo climate-control, thus improving a degree of comfort of a person who are in the target space.

8. The ion generator according to claim 1, wherein the ion generator releases the ions into the target space, thus reducing a stress of a person who are in the target space.

9. The ion generator according to claim 1, wherein the ion generator releases the ions into the target space, thus improving a degree of concentration of a person who are in the target space.

10. The ion generator according to claim 1, wherein the ion generator releases the ions into the target space, thus improving a degree of comfort of a person who are in the target space.
